(19) United States
(12) Patent Application Publication
(10) Pub. No.: US 2022/0351126 A1
(43) Pub. Date: Nov. 3, 2022

(54) A SYSTEM AND A METHOD FOR UNLOCKING ITEM/S

(71) Applicant: Carrier Corporation, Palm Beach Gardens, FL (US)

(72) Inventors: Ramakrishna Gattu, Hyderabad, Telangana (IN); Sunil Kumar Bali, Hyderabad, Telangana (IN); Luca F. Bertuccelli, Beverly, MA (US)

(21) Appl. No.: 17/251,436

(22) PCT Filed: Jun. 30, 2020
(86) PCT No.: PCT/IB2020/056162

§ 371 Date: Dec. 11, 2020

(30) Foreign Application Priority Data
Jul. 2, 2019 (IN) 201911026527

(51) Int. Cl. G06Q 10/08; H04W 4/80; H04W 64/00

(52) U.S. Cl. CPC G06Q 10/0833; H04W 4/80; H04W 64/003; G06Q 10/0832

(57) ABSTRACT

Aspects of the invention are directed towards a system and a method for unlocking items. One or more embodiments of the invention describe establishing a connection between an item and a user device through a communication channel and receiving an input on the user device to unlock the item after the establishment of the connection. One or more embodiments of the invention further disclose prompting on the user device to provide information before unlocking the item and transmitting an unlock command to the item through the communication channel for unlocking the item after receiving the information.

### TECHNICAL FIELD OF INVENTION

The present invention relates generally to monitoring of items. More particularly, the invention relates to a system and a method for unlocking an item using a user device.

### BACKGROUND OF THE INVENTION

At present, when an item is transported from a manufacturer's place to a retail store, the manufacturer of the item can track location of the item during the transit. Also, the manufacturer of the item is able to track the total number of items being transported from the place of manufacturing and also the information regarding the total number of items being transported at each location of the retail store.

However, after the delivery of the item, the manufacturer of the item is unable to track any further information regarding the item. Particularly, the manufacturer of the item is unable to know regarding the information of end consumer to whom the item is sold/administered or the end consumer who finally uses/consumes the item. The manufacturer of the item requires such information for effective billing and tracking purpose. The manufacturer would also want to know the pending stock of items in the market. Further, the existing solution for location tracking, a device can be tracked. However, such information related to the end consumer of the item requires additional infrastructure in the form of gateways or beacons which makes such tracking expensive.

In view of afore-mentioned problems in the existing solutions, there is a need of an effective system and method for tracking information of the end consumer who uses/consumes the items. There is also a need to track the information of the end consumer for effective billing by the manufacturer of the items. There is also a requirement to track the information of the end consumer for the manufacturer without using expensive hardware or any other expensive techniques. In order to solve the problems in the existing solutions, a system and a method are disclosed.

### SUMMARY OF THE INVENTION

Various embodiments of the invention describe a method and a system for unlocking an item. The invention provides a method comprising the steps of establishing a connection between the item and a user device through a communication channel. After establishing the connection, an input on the user device is received to unlock the item and before unlocking the item, a prompt is provided on the user device to provide information. After receiving the information, an unlock command is transmitted to the item through the communication channel for unlocking the item.

In an embodiment of the invention, the item advertises for the connection when a gesture is detected by an accelerometer on the item.

In other embodiment of the invention, the user device scans the connection to be established between the user device and the item and the user device sends the received information to a server after sending the unlock command to the item.

In another embodiment of the invention, the communication channel corresponds to a short-range communication channel.

In a further embodiment of the invention, the information comprises details related to a user receiving the item.

In another embodiment of the invention, the item transmits temperature data of the item to the user device indicating usability of the item.

In yet another embodiment of the invention, the item is coupled with an electronic device. Further, the electronic device comprises an accelerometer for detecting the gesture on the item, a communication unit for establishing a communication channel with the user device and for receiving the unlock command from the user device.

In another embodiment of the invention, the electronic device reports detachment of the item from the electronic device and/or a disgraceful unlocking of the item to a server. Further, the electronic device is integrated with a narrow-band internet of thing (NB-IoT) modem or category M1 (CAT-M1) modem for reporting to the server.

In yet another embodiment of the invention, a system is disclosed comprising an electronic device coupled with an item and a user device. The electronic device comprises an accelerometer adapted to detect a gesture by a user on the item and a communication unit adapted to establish a connection with a user device through a communication channel after detection of the gesture. Further, the user device comprises an interface adapted to receive an input to unlock the item after the establishment of the connection and also adapted to prompt on the interface to provide information before unlocking the item. Furthermore, the user device comprises a communication module adapted to transmit an unlock command to the item through the communication channel for unlocking the item after receiving the information from the user.

In an embodiment of the invention, the user device scans the connection to be established between the user device and the item and the user device sends the information to a server after sending the unlock command to the item.

In another embodiment of the invention, the communication channel corresponds to a short-range communication channel.

In a further embodiment of the invention, the information comprises details related to a user receiving the item.

In another embodiment of the invention, the communication unit of the electronic device is adapted to transmit temperature data of the item to the user device indicating usability of the item.

In yet another embodiment of the invention, the electronic device reports detachment of the item from the electronic device and/or a disgraceful unlocking of the item to a server. Also, the electronic device is integrated with a narrow-band internet of thing (NB-IoT) modem or category M1 (CAT-M1) modem for reporting to the server.

In a further embodiment of the invention, the communication unit of the electronic device is further adapted to receive the unlock command from the user device.

In various other embodiments of the invention, a computer readable medium is disclosed comprising one or more processors and a memory coupled to the one or more processors. The memory stores instructions which are executed to establish a connection between the item and a user device through a communication channel. After establishing the connection, an input on the user device is received to unlock the item and before unlocking the item, a prompt is provided on the user device to provide information. After receiving the information, an unlock command is transmitted to the item through the communication channel for unlocking the item.

In an embodiment of the invention, the item is coupled with an electronic device, the electronic device reports detachment of the item from the electronic device and/or a disgraceful unlocking of the item to a server using a narrow-band internet of thing (NB-IoT) modem or category M1 (CAT-M1) modem.

This summary is provided to introduce a selection of concepts in a simplified form from that are further described below in the detailed description. This summary is not intended to identify key features or essential features of the claimed subject matter, nor is it intended to be used as an aid in determining the scope of the claimed subject matter.

Other aspects, advantages, and salient features of the invention will become apparent to those skilled in the art from the following detailed description, which, taken in conjunction with the annexed drawings, discloses exemplary embodiments of the invention.

### BRIEF DESCRIPTION OF THE DRAWINGS

FIG. 1 depicts an exemplary system architecture according to an exemplary an embodiment of the invention.

FIG. 2 depicts an exemplary block diagram of different components in an electronic device coupled to an item according to an exemplary embodiment of the invention.

FIG. 3 depicts an exemplary block diagram of different components in a user device according to an exemplary embodiment of the invention.

FIG. 4 depicts an exemplary flowchart illustrating a method to perform the invention according to an exemplary embodiment of the invention.

Corresponding reference numerals indicate corresponding parts throughout the drawings.

### DETAILED DESCRIPTION OF THE INVENTION

Described herein is the technology for a system and a method of unlocking an item, the item is coupled with an electronic device. Further, the item connects with a user device through a communication channel.

As used herein, the item includes content for consumption or usage by an end customer. Such item may be a bottle or a box having liquid content, solid content or semi solid content or any such item known in the art. The content in the item may be for any kind of consumption/usage and may be a pharmaceutical content, a liquid content, an eatable content, a lotion, a cream, tablets or any such content known in the art.

As used herein, the electronic device is coupled to the item and may be a printed circuit board comprising a communication unit, a modem, an accelerometer, a processor, a memory and/or one or more sensors.

As used herein, the user device may be a desktop computer or a hand held device such as a mobile phone with a communication module, an interface, a processor, a memory and with network connectivity. Example of the user equipment includes a desktop, workstation PC, a laptop, a smart phone, a tablet, a wearable device and the like.

As used herein, the communication channel may be a channel through which the user device and the item communicates. The communication channel may be a short-range channel, a cellular channel, a long-term evolution (LTE) channel, a WiFi channel and such channel known in the art.

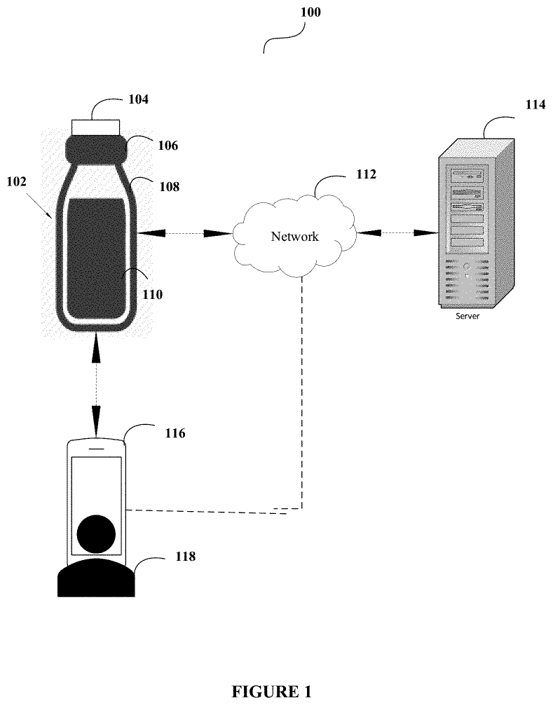

FIG. 1 depicts an exemplary system architecture 100 for unlocking an item, according to an exemplary embodiment of the invention. As depicted in FIG. 1, the system architecture 100 comprises an item 102, a user device 116 and a server 114. The item 102 is coupled with an electronic device 104. The item 102 also comprises a cap 106 to be fitted on a body 108 and content 110 in the body 108 of the item 102. The item 102 is capable of communicating with the user device 116 through a communication channel and with a server 114 over a network 112. Further, the user device 116 is capable of communicating with the server 114 through the network 112 and a user 118 is associated with the user device 116. Although the FIG. 1 depicts the electronic device 104 positioned on the cap 106 of the item 102, however, it is understood for a person skilled in the art that the electronic device 104 can be positioned anywhere on the item 102.

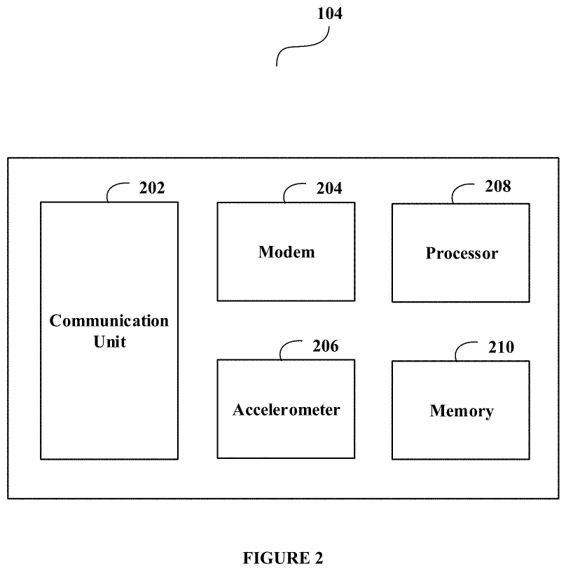

FIG. 2 depicts an exemplary block diagram of different components in the electronic device 104 coupled to the item 102, according to an exemplary embodiment of the invention. The electronic device 104 is coupled with the item 102 and comprises a communication unit 202, a modem 204, an accelerometer 206, a processor 208, and a memory 210. The accelerometer 206 is adapted to detect a gesture made by the user 118 on the item 102. Such gesture may include opening the cap 106 of the item 102, pressing the cap 106, tapping (single tap or double tap) on the item 102, shaking the item 102, holding the item 102 or any such gesture made by the user on the item 102 and recognized by the accelerometer 206 therein. After detection of the gesture by the accelerometer 206, the communication unit 202 of the electronic device 104 is adapted to advertise the item 102 or the electronic device 104. The advertised item 102 can be recognized by the user device 116, where the user 118 can input credentials to establish a connection over the communication channel. The advertisement of the item 102 can be active as long as the communication unit 202 is active. In another embodiment of the invention, the communication unit 202 may be active for a short duration of time (may be for 30 seconds or 1 minute). The communication unit 202 of the item 102 is adapted to establish connections from the user device 116 when appropriate credentials are provided. If the duration for advertisement by the communication unit 202 expires, the item 102 stops advertising until the user 118 made another gesture on the item 102.

When the communication unit 202 advertises the item 102, the user 118 of the user device 116 may open an application stored in the user device 116 and scans for the item 102 in the application. The application also lists the item 102 along with an identifier of the item 102. As used herein, the application in the user device 116 is provided by a manufacturer of the item 102. Then, the user 118 can select the item 102 in the application for establishing a connection using the credentials as discussed above. The communication unit 202 of the electronic device 104 establishes a connection between the item 102 and a user device 116 through a communication channel. Such communication channel may correspond to a short-range communication channel like a bluetooth channel in a personal area network. Further, the communication channel is a secure channel and avoids malicious attacks or un-authored access.

Once the connection between the item 102 and the user device 116 is established through the communication channel, the user 118 of the user device 116 provide an input in the application of the user device 116 to unlock the item 102. Such input can be provided by tapping or clicking on an unlock tab provided in the application. When the user 118 provides the input to unlock the item 102, the application of the user device 116 prompts the user 118 of the user device 116 to provide information. Such information may be in the form of details related to the user 118 who wishes to use/consume the content 110 of the item 102 or may be related to any person who wishes to use/consume the content 110 of the item 102. The information may include, but is not limited to, a name of the user, an age of the user, a location of the user, and such information well known in the art. Alternatively, the application may display a form which the user 118 may fill to provide the information. Such information can also provide through voice commands by the user 118. The information may vary from manufacturer to manufacturer and may depend upon the type of item being consumed. Moreover, the information may be limited as per the requirement of a particular manufacturer.

After the user 118 provides information to the application in the user device 116, the user device 116 transmits an unlock command to the item 202 through the communication channel for unlocking the item 102. The communication unit 202 of the electronic device 104 (coupled to the item) receives the unlock command from the user device 116 and thereby, the item 102 can be unlocked. Further, the user device 116 transmits the information provided by the user 118 to the server 114, which is accessed by the manufacturer of the item 102. Once the item 102 is unlocked, the application in the user device 116 can display health of the item 102. For example, the item 102 is good to use or not.

Further, the item 102 encompasses one or more temperature sensors to sense environment temperature in which the item 102 is placed/situated. Based on sensing the temperature, the communication unit 202 of the electronic device 104 transmits the sensed temperature data of the item 102 to the user device 116. Also, the application in the user device 116 displays an alarm, for an example, when the temperature of the item 102 does not meet health requirements. Thereby, the user device 116 through the application indicates to the user 118 whether the item 102 is usable or not. In alternative embodiment of the invention, the item 102 transmits sensed temperature data of the item 102 to the server 114.

When the item 102 is unlocked by the user 118 without providing the information, then in such a scenario, the modem 204 of the electronic device 104 reports this accidental or disgraceful unlocking of the item 102 to the server 114. The modem 204 can be a Narrow-band Internet-of-Thing (NB-IoT)/Category (CAT) M1 modem. For accidental or disgraceful unlocking of the item 102, the user 118 would have to manually break a lock of the item 102. Also, the modem 204 of the electronic device 104 reports the server 114 when the item 102 is detached from the electronic device 104 for the purpose of accidental or disgraceful unlocking of the item 102. The memory 210 may be adapted to store the identifier of the item 102, details related to connections established etc.

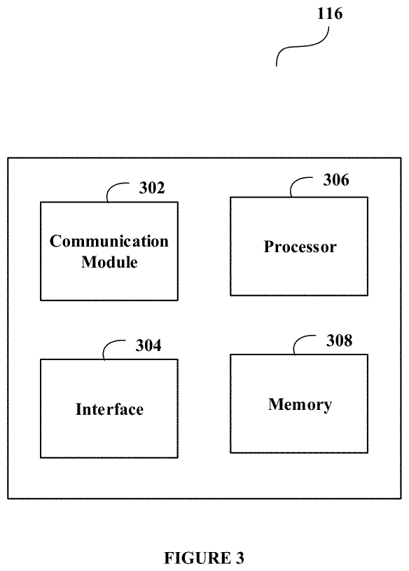

FIG. 3 depicts an exemplary block diagram of different components in the user device 116, according to an exemplary embodiment of the invention. The user device 116 comprises, but is not limited to, a communication module 302, an interface 304, a processor 306 and a memory 308. The communication module 302 is adapted to establish a connection with the item 102 through a communication channel as discussed above. Such communication channel corresponds to a short-range communication channel like a bluetooth channel in a personal area network. After the connection is established with the item 102 through the communication channel, the interface 304 is adapted to receive an input to unlock the item 102 and the interface 304 is also adapted to prompt the user to provide information before unlocking the item 102. After receiving the information from the user, the communication module 302 is adapted to transmit an unlock command to the item 102 through the communication channel. Then, the communication unit 202 of the electronic device 104 receives the unlock command from the user device 116 and thereby, the item 102 can be unlocked. The memory 308 is adapted to store the application of the manufacturer.

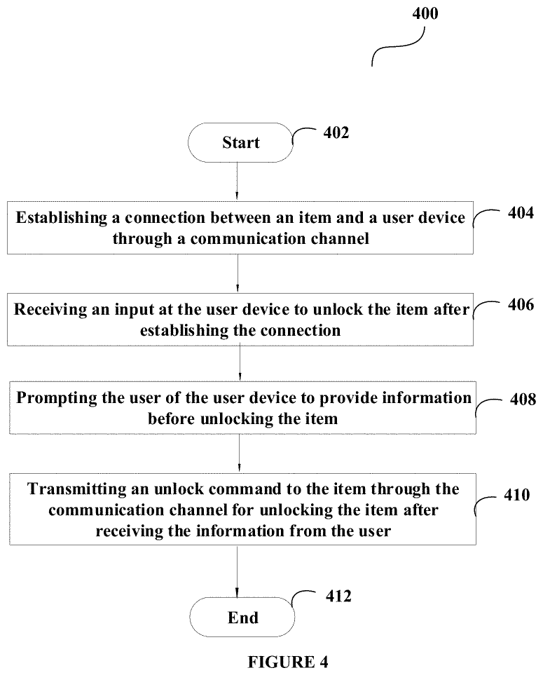

FIG. 4 depicts a flowchart outlining the features of the invention in an exemplary embodiment of the invention. The method flowchart 400 describes a method being performed for enabling the invention. The method flowchart 400 starts at step 402.

At step 404, a connection is established between the item 102 and the user device 116 through a communication channel. Such communication channel corresponds to a short-range communication channel like a bluetooth channel in a personal area network.

At step 406, after the connection is established with the item 102 through the communication channel, an input is received to unlock the item 102. Such input can be provided by tapping or clicking on an unlock tab provided in the application.

At step 408, the user 118 is prompted to provide information before unlocking the item 102.

At step 408, after receiving the information from the user, an unlock command is transmitted to the item 102 through the communication channel. Then, the communication unit 202 of the electronic device 104 receives the unlock command from the user device 116 and thereby, the item 102 can be unlocked. The method flowchart ends at 412.

The present invention also encompasses an electric lock and/or electro-mechanic lock on the item 102, wherein the electric lock may be a soft lock and the electro-mechanic lock may be a hard lock. The soft lock can be automatically unlocked when the unlock command is received by the item 102 from the user device 116. The hard lock can then be manually unlocked by the user 118 of the item 102.

The present invention is applicable in various fields such as, but not limited to, pharmaceutical industry, cosmetics industry, food industry and any such field that is well known in the art.

The present invention provides the following technical advantages over the existing methods and systems: a) effective billing and tracking of users of the items by the manufacturers of the items, b) enabling manufacturers of the item to receive information regarding the users of the items, c) enabling manufacturers to receive user information through an application of the manufacturers, d) reporting disgraceful/accidental unlocking of the item to the manufacturers, e) informing consumer/user about the health of the item before unlocking the item, and f) enabling users of the item to verify integrity of the content of the item based on health of the item.

The embodiments of the invention discussed herein are exemplary and various modification and alterations to a person skilled in the art are within the scope of the invention.

In one embodiment of the invention, the invention can be operated using the one or more computer readable devices. The one or more computer readable devices can be associated with the item 102. The computer readable medium of the computer readable devices is configured to establish the connection between the item 102 and the user device 116 through the communication channel. The computer readable medium of the computer readable devices is also configured to receive the input at the user device 116 to unlock the item 102 after establishing the connection and prompt the user 118 of the user device 116 to provide information before unlocking the item 102. The computer readable medium of the computer readable devices is further configured to transmit the unlock command to the item 102 through the communication channel for unlocking the item 102 after receiving the information from the user 118. Furthermore, the item 102 is coupled with the electronic device 104. The electronic device 104 reports detachment of the item from the electronic device and/or a disgraceful unlocking of the item to a server using a narrow-band internet of thing (NB-IoT) modem or category M1 (CAT-M1) modem 206.

Exemplary computer readable media includes flash memory drives, digital versatile discs (DVDs), compact discs (CDs), floppy disks, and tape cassettes. By way of example and not limitation, computer readable media comprise computer storage media and communication media. Computer storage media include volatile and nonvolatile, removable and non-removable media implemented in any method or technology for storage of information such as computer readable instructions, data structures, program modules or other data. Computer storage media are tangible and mutually exclusive to communication media. Computer storage media are implemented in hardware and exclude carrier waves and propagated signals. Computer storage media for purposes of this invention are not signals per se. Exemplary computer storage media include hard disks, flash drives, and other solid-state memory. In contrast, communication media typically embody computer readable instructions, data structures, program modules, or other data in a modulated data signal such as a carrier wave or other transport mechanism and include any information delivery media.

Although described in connection with an exemplary computing system environment, examples of the invention are capable of implementation with numerous other general purpose or special purpose computing system environments, configurations, or devices.

Examples of the invention may be described in the general context of computer-executable instructions, such as program modules, executed by one or more computers or other devices in software, firmware, hardware, or a combination thereof. The computer-executable instructions may be organized into one or more computer-executable components or modules. Generally, program modules include, but are not limited to, routines, programs, objects, components, and data structures that perform particular tasks or implement particular abstract data types. Aspects of the invention may be implemented with any number and organization of such components or modules. For example, aspects of the invention are not limited to the specific computer-executable instructions or the specific components or modules illustrated in the Figures and described herein. Other examples of the invention may include different computer-executable instructions or components having more or less functionality than illustrated and described herein.

Aspects of the invention transform a general-purpose computer into a special-purpose computing device when configured to execute the instructions described herein.

The order of execution or performance of the operations in examples of the invention illustrated and described herein is not essential, unless otherwise specified. That is, the operations may be performed in any order, unless otherwise specified, and examples of the invention may include additional or fewer operations than those disclosed herein. For example, it is contemplated that executing or performing a particular operation before, contemporaneously with, or after another operation is within the scope of aspects of the invention.

When introducing elements of aspects of the invention or the examples thereof, the articles “a,” “an,” “the,” and “said” are intended to mean that there are one or more of the elements. The terms “comprising,” “including,” and “having” are intended to be inclusive and mean that there may be additional elements other than the listed elements. The term “exemplary” is intended to mean “an example of” The phrase “one or more of the following: A, B, and C” means “at least one of A and/or at least one of B and/or at least one of C”.

Having described aspects of the invention in detail, it will be apparent that modifications and variations are possible without departing from the scope of aspects of the invention as defined in the appended claims. As various changes could be made in the above constructions, products, and methods without departing from the scope of aspects of the invention, it is intended that all matter contained in the above description and shown in the accompanying drawings shall be interpreted as illustrative and not in a limiting sense.

Although the subject matter has been described in language specific to structural features and/or acts, it is to be understood that the subject matter defined in the appended claims is not necessarily limited to the specific features or acts described above. Rather, the specific features and acts described above are disclosed as examples of implementing the claims and other equivalent features and acts are intended to be within the scope of the claims.

1. A method for unlocking an item, the method comprising:
establishing a connection between the item and a user device through a communication channel;
receiving an input on the user device to unlock the item after the establishment of the connection;
prompting on the user device to provide information before unlocking the item; and
transmitting an unlock command to the item through the communication channel for unlocking the item after receiving the information.

2. The method of claim 1, wherein the item advertises for the connection when a gesture is detected by an accelerometer on the item.

3. The method of claim 1, wherein the user device scans the connection to be established between the user device and the item and the user device sends the received information to a server after sending the unlock command to the item.

4. The method of claim 1, wherein the communication channel corresponds to a short-range communication channel.

5. The method of claim 1, wherein the information comprises details related to a user receiving the item.

6. The method of claim 1, wherein the item transmits temperature data of the item to the user device indicating usability of the item.

7. The method of claim 1, wherein the item is coupled with an electronic device.

8. The method of claim 7, wherein the electronic device coupled to the item comprises:
an accelerometer for detecting the gesture on the item;
a communication unit for establishing a communication channel with the user device and receiving the unlock command from the user device.

9. The method of claim 7, wherein the electronic device reports detachment of the item from the electronic device and/or a disgraceful unlocking of the item to a server.

10. The method of claim 9, wherein the electronic device is integrated with a narrow-band internet of thing (NB-IoT) modem or category M1 (CAT-M1) modem for reporting to the server.

11. A system comprising:
an electronic device coupled with an item, the electronic device comprising:
an accelerometer adapted to detect a gesture by a user on the item; and
a communication unit adapted to establish a connection with a user device through a communication channel after detection of the gesture; and
a user device comprising:
an interface adapted to:
receive an input to unlock the item after the establishment of the connection; and
prompt on the interface to provide information before unlocking the item; and
a communication module adapted to transmit an unlock command to the item through the communication channel for unlocking the item after receiving the information from the user.

12. The system of claim 11, wherein the user device scans the connection to be established between the user device and the item and the user device sends the information to a server after sending the unlock command to the item.

13. The system of claim 11, wherein the communication channel corresponds to a short-range communication channel.

14. The system of claim 11, wherein the information comprises details related to a user receiving the item.

15. The system of claim 11, wherein the communication unit of the electronic device is adapted to transmit temperature data of the item to the user device indicating usability of the item.

16. The system of claim 11, wherein the electronic device reports detachment of the item from the electronic device and/or a disgraceful unlocking of the item to a server.

17. The system of claim 16, wherein the electronic device is integrated with a narrow-band internet of thing (NB-IoT) modem or category M1 (CAT-M1) modem for reporting to the server.

18. The system of claim 11, wherein the communication unit of the electronic device is further adapted to receive the unlock command from the user device.

19. A computer readable medium comprising one or more processors and a memory coupled to the one or more processors, the memory storing instructions which are executed by the one or more processors, the one or more processors configured to:
establish a connection between the item and a user device through a communication channel;
receive an input on the user device to unlock the item after establishing the connection;
prompt on the user device to provide information before unlocking the item; and
transmit an unlock command to the item through the communication channel for unlocking the item after receiving the information.

20. The computer readable medium of claim 19, wherein the item is coupled with an electronic device, the electronic device reports detachment of the item from the electronic device and/or a disgraceful unlocking of the item to a server using a narrow-band internet of thing (NB-IoT) modem or category M1 (CAT-M1) modem.
